(19) United States
(12) Patent Application Publication
(10) Pub. No.: US 2009/0028969 A1
(43) Pub. Date: Jan. 29, 2009

(54) Compostion for treating skin

(76) Inventors: Gerard Sene, Paris (FR); Alain Loiseau, Bouillon (FR); Virginie Petit, Pau (FR); Eric Theron, Montardon (FR); Caroline Segond, Bernadets (FR)

Correspondence Address: BAYER HEALTHCARE LLC;CONSUMER CARE DIVISION, 36 COLUMBIA ROAD, MORRISTOWN, NJ, 07962, US

(21) Appl. No.: 12/229,621

(22) Filed: Aug. 26, 2008

(30) Foreign Application Priority Data
Feb. 28, 2006 (EP) 06290343.0
Feb. 21, 2007 (EP) PCT/EP2007/001459

(51) Int. Cl. A61K 36/484; A61K 31/7034; A61P 17/00

(52) U.S. Cl. 424/757; 514/25

(57) ABSTRACT

A combination comprising verbascoside and luteolin, and its use in a cosmetic or pharmaceutical composition for pigmentation modulation.

### 1. FIELD OF THE INVENTION

The present invention relates to a combination comprising verbascoside and luteolin, a plant extract comprising the combination and the use of such combinations in a cosmetic or pharmaceutical composition useful for modulation of skin pigmentation.

### 2. BACKGROUND OF THE INVENTION

The caffeic acid derivative Verbascoside is an ortho-dihydroxycinnamic acid derivative of a phenylpropanoid glycoside. Phenylpropanoid glycosides are known for their therapeutic properties in many formulations such as anti-fungal, anti-bacterial, anti-viral, analgesic formulations.

The chemical name of Verbascoside is 2-(3′,4′-dihydroxyphenyl)ethyl-O-α-1-rhamnopyranosyl-(1→3)-β-d-(4-O-caffeoyl)-glucopyranoside and its complete structure was elucidated in 1963 under the name acteoside (Birkofer et al, Z Naturforsch B, 1968, 23(8), 1051-8). Verbascoside is also called Kusaginin and its use is already known in cosmetics.

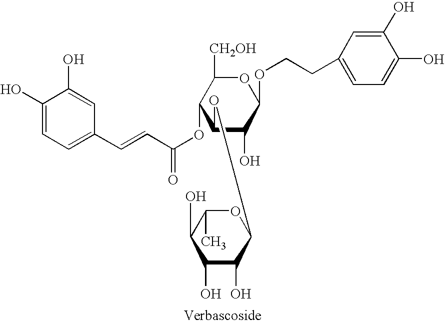

The use of verbascoside in anti-ageing cosmetic compositions is described in PCT Application No. PCT/FR2004/000252 to Robin et al., filed Feb. 3, 2004 and published Apr. 19, 2004 (Publication No. WO2004/069218). Verbascoside was disclosed as being useful to stimulate synthesis of stress proteins (HSP 70) by skin cells and to enable the skin to defend efficiently against environmental aggression. The application (WO2004/069218) also described the extraction of verbascoside from plants of the Tubiflorae order and more specifically from plants of the genus Verbascum, Pladago, Verbena, Lippia or Fraxymus.

A poster entitled, “The effect of verbascoside, an extract of Chinese herbal medicine on formation of free radicals in brain and skeletal muscle after exhaustive exercice,” (K. M. Chan & J. X. Li) presented at the 5th IOC World Congress 1999 on Sport Sciences presents the anti-free radicals effect of verbascoside.

Verbascoside is discussed as an active compound for skin whitening in Japanese Patent Specification JP 2005-082522, and In WO 01/026670 extracts from olive plants containing verbascoside are described for anti-aging and skin whitening activity.

Verbascoside is commercially available and methods for verbascoside extraction have already been described. Verbascoside can be obtained from different plants, for example from Scrophulariaceae, Piperaceae, Labiatae, Acanthaceae or Orobranchaceae such as Pedicularis sp. (CN1291613), Piper aduncum (JP2000302797), Leucosceptrum sp (JP2191292), Orobranche hedera (FR2302745).

Complexion coloration is hormonally and genetically determined but pigmentation changes occur in response to UV radiation. After UV induction skin color is primarily regulated by melanogenesis. This complex biochemical chain reaction takes place in epidermis and corresponds to the melanin pigment production by the dendritic melanocytes. Melanin comprises two classes of polypeptides: the reddish-yellow phaeomelanin and the dark brown eumelanin. Melanogenesis is influenced by specific mediators—like the tyrosinase enzyme and the tyrosinase-related proteins (TRP1, TRP2)—which contribute to define the melanin amount and the type of the melanin pigment and therefore participate to skin complexion (Petit L, Piérard G E, Int J Cosmet Sci, 2003, 25(4), p. 169-181).

In addition, contributing factors for skin color comprise efficient transfer of melanin from the melanocytes to the neighboring keratinocytes and distribution and degradation of the transferred melanosomes by the recipient keratinocytes (Boissy R E, Exp Dermatol. 2003; 12 Suppl 2:5-12). Playing a role in the melanocyte dendrification and/or in the melanin-containing organelles (melanosomes) this transfer also participate to pigmentation modulation.

Luteolin is a flavonoid molecule, which chemical name is 3′,4′,5,7-Tetrahydroxyflavone.

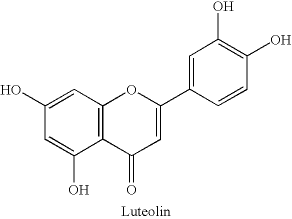

Luteolin is known for its activity on pigmentation.

FR2578422 claims a topical treatment with a biologically active amount of luteolin. The composition is said to be active for hypermelanized spots treatment without toxicity problem. Luteolin can be extracted e.g. from the dried aerial part of Achillea millefolium.

Luteolin is also mentioned to be anti-oxidant. DE19962345 describes a cosmetic composition with anti-oxidant property comprising a Arachis hypogaea seeds extract containing at least 50% of luteolin. EP 1072265 displays the use of luteolin in combination with other polyphenolic compounds for anti-oxidant activity.

Arbutin is known for its activity on melanogenesis due to tyrosinase inhibition (Maeda K, Fukuda M, J. Pharmacol. Exp. Ther., 1996, 276, 765-769; Chakraborty and al, Pigment Cell Res, 1998, 11(4), 206-12). This hydroquinone β-d-glucopyranoside is therefore often used as a reference in enzymatic, cell cultures or in vivo substantiation tests. For example, in the enzymatic mushroom tyrosinase assay which is currently used as screening test for whitening property, the anti-tyrosinase IC50 for arbutin is about 100 μg/ml (Lee K T et al., Int J Cosmet Sci, 1997, 19(6), 291-98; Kang H S et al, Arch Pharm Res, 2004, 27(12), 1226-32; Funamyama M et al, Bioscience, Biotechnology and Biochemistry, 1995, 59(1), 143-44). This compound is used as such or derived from plant—e.g. from Uvea ursi folium (Petit L, Piérard, G. E., Int J Cosmet Sci, 2003, 25(4), p. 169-81)—in lightening cosmetic products as this hydroquinone derivative is safer than hydroquinone, which is forbidden in cosmetics owing to its cytotoxicity.

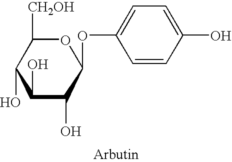

The Buddlejaceae plant family consists of nine genera (Androya, Buddleja, Emorya, Gomphostigma, Nicodemia, Nuxia, Peltanthera) and about 150 species.

Buddleja axillaris Willd, also called Adenoplusia axillaris, is a shrub, which is 2 to 5 m high and grows mainly in secondary forests in Madagascar and in East Africa. The opposite leaves are simple, petiolate to sessile, 7-12 cm long, 2-4.5 cm wide; the limb upper surface is green and slightly hairy, whitish and tomentose on its lower surface. The flowers are terminal, thyrsoid cymes with white corolla, densely tomentose externally and glabrous within. The fruit is brown, fleshy, globose, indehiscent and about 2.5 mm in diameter. The seeds are ellipsoid and about 1 mm long. In Madagascar this plant is locally named ‘Sevafotsy’ or ‘Mandresy’ and is traditionally used for healthcare e.g. the aqueous decoction is used as beverage for headaches treatment and a mixture comprising an aqueous decoction of leaves and bark combined with some boiled plant is used as cataplasm against rheumatism and arthrosis.

There are Japanese patents describing Buddleja coriacea extracts for its use alone or in combination with another plant extract in whitening compositions (JP5225062, JP8012565). A specific flavonoid molecule, called Buddlenoid, is disclosed as active compound (JP5255376).

Buddleja officinalis has also been studied. Four flavonoids, one phenylethyl glucoside and one phenylpropanoid glycoside were isolated from the flowers of Buddleja officinalis. Among these molecules, luteolin and acteoside (=verbascoside) were shown to have antioxidant property (Piao M S, Kim M R, Lee D D G, Park Y, Hahm K S, Moon Y H, Woo E R, Arch Pharm Res, 2003 June, 26(6), 453-7).

French Patent No. 2,831,444 refers to a cosmetic or dermatological composition comprising hydrosoluble extracts of Buddleja davidii and Anthyllis vulneraria. This composition is claimed to have moisturizing, soothing, anti-irritation and wound healing properties for skin repair after sun exposure. The hydrosoluble Buddleja extract composition is described and contains iridoids, flavonoids, caffeic acid esters and triterpenoids.

Verbascoside has already been isolated and identified in other species of the Buddlejaeceae family, for example from Buddleja yunanesis (Liao, Y. H. et al, J Nat Prod, 1999, 62(9), 1241-45) or from Buddleja purdomii (Gao, Y. et al, Zhong Yao Cai, 2004, 27(5), 339-41). The isolated verbascoside from Buddleja cordata (Avila Acevedo, J. C. et al, Fitoterapia, 1999, 66(1), 75-78) and from Buddleja globosa leaves (Pardo, F. et al, J. of Ethnopharmacology, 1993, 39(3), 221-22) has been shown to have anti-bacterial activity. Furthermore, verbascoside can be isolated from Buddleja scordioides (Avila Acevedo, J. C. et al, Fitoterapia, 2005, 76(3-4), 301-09).

The invention relates to a combination comprising verbascoside and luteolin, and/or a plant extract containing the combination for pigmentation modulation. The use of the combination according to the invention and the extract containing the combination are an appropriate and safe method for pigmentation modulation of skin.

Plant extracts containing verbascoside according to the invention are extracts of plants which include but are not limited to the Tubiflorae plant family comprising, e.g., the Verbascum, Pladago, Verbena, Lippia or Fraxymus genus; the Buddlejaceae plant family comprising, e.g., the Androya, Buddleja, Emorya, Gomphostigma, Nicodemia, Nuxia or Peltanthera genus; or the Labiatae plant family comprising e.g. Ballota, Faradaya genus. Preference is given to the Buddleja genus and more preferably the plant extract is an extract of Buddleja axillaris.

The extraction can be performed on all parts of the plant(s). Preferably, however, the extraction is performed on leaves of Buddleja axillaris.

The extraction can be carried out using standard extraction methods. Preferably, the extraction is carried out with a polar solvent suitable for extraction. Leaves may be extracted with a polar solvent. The extraction may be carried out several times to increase the amount of material extracted. The solution obtained is then mixed and extracted with a non polar solvent e.g., heptane, to remove the waxes, essential oils, pigments and most of the non polar molecules. After phase separation the solvent of the remaining polar phase is removed in order to obtain a dry extract containing verbascoside. Optionally, the extract can be dried by adding water and conducting a freeze-drying.

An extract according to the invention is normally a dry extract. The extract can also be used as solution, in which case the final drying step of the extraction process may be omitted.

The polar solvent used for extraction is preferably alcohol or a mixture of water and alcohol wherein the alcohol is preferably ethanol. The volume ratio of the water and alcohol can be from about 50:50 up to about 90:10, and is preferably about 70:30.

A dry plant extract containing verbascoside in an amount of more than 10%, preferably more than 15%, most preferably 16% to 25%, and luteolin in an amount of up to 5%, more preferably up to 2%, most preferably up to 1% by weight of the total plant extract is preferred. The plant extract contains should contain luteolin in an amount of at least 0.01% by weight of the total plant extract. Most preferably the plant extract is an extract of Buddleja axillaris.

Surprisingly, only a small amount of luteolin in combination with verbascoside provides an improved pigmentation alteration effect compared to the administration of verbascoside alone. The combination can be synergistic, e.g., where the joint action of the drugs is such that the combined effect is greater than the algebraic sum of their individual effects. Thus, reduced amounts of the drugs can be administered, e.g., reducing toxicity or other deleterious or unwanted effects, and/or using the same amounts as used when the agents are administered alone, but achieving greater efficacy. The reduced amounts of the drugs can be lower then used in a standard method wherein, e.g., the single drug is administered.

The combination of the invention can be administered at any time and in any effective form. For example, the compounds can be administered simultaneously, e.g., as a single composition or dosage unit (e.g., a pill or liquid containing both compositions), or they can be administered as separate compositions, but at the same time (e.g., where one drug is administered intravenously and the other is administered orally or intramuscularly). The drugs can also be administered sequentially at different times. Agents can be formulated using conventional techniques to achieve the desired rates of release over extended period of times, e.g., 12 or 24 hours. Extended or sustained release can be achieved by using agents and/or derivatives that have suitable metabolic half-lives, and/or by using controlled release formulations.

The combination of verbascoside and luteolin can also be isolated and/or purified from the extract containing it by standard isolation methods. Standard isolation methods include but are not limited to chromatographic methods.

The combination or the extract containing it can be administered in any form by any effective route, including, e.g., oral, parenteral, enteral, intravenous, intraperitoneal, topical, transdermal (e.g., using any standard patch), ophthalmic, nasally, local, non-oral, such as aerosal, inhalation, subcutaneous, intramuscular, buccal, sublingual, rectal, vaginal, intra-arterial, and intrathecal, etc. The combination can be administered alone, or in combination with any other ingredient(s), active or inactive. Topical administration is preferred.

The combination or the extract containing it can be converted in a known manner into formulations such as cosmetic, dermatologic and/or pharmaceutical compositions. These formulations may be liquid or solid, e.g., normal and enteric coated tablets, capsules, pills, powders, granules, elixirs, tinctures, solution, suspensions, suppositories, syrups, solid and liquid aerosols, emulsions, pastes, creams, ointments, milks, gels, salves, serums, foams, shampoos, sticks, lotions or any other form acceptable for administration. Dermatological or cosmetic compositions in the form of an aqueous solution, a white or colored cream, ointment, milk, gel, salve, serum, foam, shampoo, stick, cream, paste, or lotion are preferred.

The combination or the extract containing it can be further combined with any other suitable additive or pharmaceutically acceptable carrier, preferably one or more dermatological and/or cosmetically acceptable carriers. Such additives include any of the substances already mentioned, as well as any of those used conventionally, such as those described in Remington, The Science and Practice of Pharmacy (Gennaro and Gennaro, eds, 20th edition, Lippincott Williams & Wilkins, 2000), in Theory and Practice of Industrial Pharmacy (Lachman et al., eds., 3rd edition, Lippincott Williams & Wilkins, 1986), and in Encyclopedia of Pharmaceutical Technology (Swarbrick and Boylan, eds., 2nd edition, Marcel Dekker, 2002). Such materials are referred to herein as “pharmaceutically acceptable carriers” to indicate they are combined with the active drug and can be administered safely to a subject for therapeutic purposes.

The dosage of the combination or the extract containing it of the present invention can be selected with reference to the effects to be treated and/or the type of disease and/or the disease status in order to provide the desired therapeutic activity. These amounts can be determined routinely for a particular patient, where various parameters are utilized to select the appropriate dosage (e.g., type of disease, age of patient, disease status, patient health, weight, etc.), or the amounts can be relatively standard.

The amount of the administered active ingredients can vary widely according to such considerations as the particular compound and dosage unit employed, the mode and time of administration, the period of treatment, the age, sex, and general condition of the patient treated, the nature and extent of the condition treated, the rate of drug metabolism and excretion, the potential drug combinations and drug-drug interactions, and the like.

Preferably, verbascoside should be present in a composition in an amount of at least about 0.0001% by weight and preferably at least about 0.001% by weight of the total composition. Verbascoside may comprise up to about 10% by weight, preferably up to about 5% by weight, and more preferably up to about 1% by weight of the total composition. Luteolin may comprise up to about 1% by weight, more preferably up to about 0.1% by weight, and most preferably up to 0.05% by weight of the total composition. Luteolin is preferably present in an amount of at least about 0.00001% and more preferably up to about 1% by weight of the total composition.

When the dry plant extract is used it should preferably be present in the composition in an amount of from about 0.01% to about 10% by weight and preferably from about 0.1% to about 1% by weight of the total composition.

The composition may be administered one or more times a day, more preferably up to three times a day, and most preferably two times per day. Topical administration is preferred.

It may in some cases be advantageous to deviate from the amounts and dosage level described herein, depending on factors such as the body weight of the patient, individual tolerance for and compatibility with the active ingredient, the type of preparation and time or interval over which the administration is effected. For instance, less than the aforementioned minimum amounts may be sufficient in some cases, while the upper limit specified may be exceeded in other cases. In the case of administration of relatively large amounts, it may be advisable to divide the daily dosage into several individual doses administered throughout the day.

The combination of the invention or the extract containing it can also be combined with at least one other active substance or extract containing that substance usually employed for dermatological use. Other active substances include but are not limited to substances for whitening of the skin, lightening of the skin, spots prevention or treatment of spots, e.g., hydroquinone, tretinoin, topical steroids, azelic acid, kojic acid, arbutin, luteolin, licorice and extracts containing these substances may be used. Arbutin and luteolin and extracts containing these substances are preferred.

Substances relevant for pigmentation modulation like UV sunscreens or filters or keratolytic agents such as alpha hydroxyacids may also be combined with the combination of the invention or an extract containing the combination.

The combination of the invention or an extract containing it may be used in the dermatological field, which includes cosmetic and pharmaceutic use for pigmentation modulation. The combination of the invention or an extract containing it may be used cosmetically for whitening of the skin, lightening of the skin, prevention or reduction of pigmentation spots of the skin (age-related or photo-induced spots), anti-pigmentation of the skin, unifying skin tone and/or fair skin.

The combination of the invention or an extract containing it may also be used for the treatment, prevention or regulation of pigmentation disorders, which include, but are not limited to, post-inflammatory hyperpigmentation after wound healing (acne, eczema, contact dermatitis etc.), photomelanosis, endocrine abnormalities and pregnancy (naevus), Adison's disease, acanthose nigricans, ephelis, melasma, secondary effects of antibodies, antimalaric treatments, prevention of self protection of cancerous cells during skin treatments, progressive pigmentation purpuras, prevention of age spots and pigmentation due to the administration of cosmetics (e.g. fragrance, etc.).

The combination of the invention or an extract containing it shows activity in influencing tyrosinase, melanogenesis, UV-induced pigmentation, melanocyte dendrite formation and/or melanosomes transfer which are relevant for pigmentation modulation.

The following examples serve to illustrate aspects of the invention but are not intended to limit the scope of the invention, which is defined by the claims appended hereto.

### EXAMPLE 1

### Preparation of Buddleja Axillaris Extracts

Crushed dry leaves of Buddleja axillaris were extracted with a mixture of ethanol and water in a volume ratio of 70:30. The solution was stirred and heated during the extraction step. The solid material was filtered off and the extraction was repeated several times. The extraction time was between 30 minutes and 1 hour. The temperature was below 60° C. The combined alcoholic extracts were then mixed and extracted with heptane. After phase separation the remaining polar phase was distilled under vacuum to remove the solvent. (Optionally water may be added to enable a freeze-drying to obtain the final dry extract containing verbascoside.)

The final dry extract was characterized by thin layer chromatography and HPLC standard method. The final extract showed a content of 19% of verbascoside and 0.1% luteolin by weight of the total dry extract.

### EXAMPLE 2

### Tyrosinase Inhibitory Activity

The inhibitory activity of the Buddleja axillaris extract produced according to Example 1 was evaluated in vitro. The method was based on the determination of the dopa-oxidase activity of mushroom tyrosinase by measuring the increase in the absorbance at 475 nm due to dopachrome function photometrically.

The inhibitory concentration (“IC50”) is defined as the concentration of the test product which reduces the dopa oxidase activity of the control tyrosinase by 50%. This value is calculated and the data is expressed in variation of absorbance per minute (ΔA/Δt) in the photometric measurement.

Buddleja extract was dissolved directly in the assay buffer (phosphate buffer). Five concentrations were tested: 0.03 mg/ml, 0.10 mg/ml, 0.30 mg/ml, 1 mg/ml, and 3 mg/ml. Each experimental condition was run in duplicate. The experimental parameters were: enzyme concentration of 40 U/ml and measurement of absorbance at 475 nm during 4 minutes.

The inhibition of tyrosinase by the product is shown in Table 1. Taking into account the linear relationship between the percentage of inhibition and the test product concentration (expressed in log), the inhibitory dose (IC50) is calculated from the regression curve:

% inhibition=32.57 log(concentration)+67.43(N=5ddl,r=0.976)

The IC50 of the test product was 290 μg/ml.

TABLE 1

| Concentration (mg/ml) | ΔA/Δt | Tyrosinase (U/ml) | % Inhibition |
|---|---|---|---|
| 0 | 0.151 | 58.6 | — |
| 0.03 | 0.126 | 48.6 | 16.9% |
| 0.1 | 0.092 | 35.5 | 39.4% |
| 0.3 | 0.076 | 29.5 | 49.6% |
| 1.0 | 0.063 | 24.2 | 58.7% |
| 3.0 | 0.017 | 6.6 | 88.8% |

### EXAMPLE 3

### Effects of Buddleja Axillaris Extract on the Cutaneous Pigmentary System

In order to evaluate the activity on pigmentation, an in vitro experiment, based on the measurement of intracellular melanin content of human cultured melanocytes exposed (UVB-stimulated) or not exposed to radiation, in the absence and in the presence of the test compound, was conducted. Buddleja axillaris extracts (extracted in accordance with Example 1), containing Verbascoside (>90% pure, Extrasynthese) and Luteolin (isolated from Buddleja axillaris) were tested.

Normal human epidermal melanocytes from newborn foreskin were cultured at 30° C. in MGM “serum free” medium (Melanocyte Growth Medium, PromoCell®) supplemented with antibiotics. Cultures were maintained at 37° C. in a humidified 5% CO₂ atmosphere.

Subcultures of human melanocytes were propaged in MGM medium and used just before reaching confluence. Cells were counted and diluted to the desired concentration in culture medium without calf serum. The cultured melanocytes were placed in 24-well plates at a density of 60×103 cells per well. Two plates were seeded with cells: one for the measurement of melanin content (“Melanin plate”) and the other for the measurement of cell densities (“Neutral Red” plate).

Treatment with UVB:

72 hours after plating, the medium was removed and replaced by fresh medium containing the test product at various non toxic concentrations. Cells were incubated at 37° C., in a 95% air-5% CO₂ atmosphere for eight days. During this period cells were exposed to UVB radiation at day 1 (D1), day 2 (D2), day 3 (D3), day 4 (D4), day 7 (D7) and day 8 (D8). UVB radiation exposure was carried out with a parallel bank of TL20W/12 tubes emitting a continuous spectrum between 280 and 320 nm with a peak emission at 312 nm. A UVB dose of 40 mJ/cm² was applied at each exposure. Sham-control cells were subjected to the same procedure, but without UV exposure and without treatment.

Before exposure, cell monolayers were washed with pre-warmed Phosphate Buffer Solution (PBS) and then exposed to UVB in the presence of PBS (without test product). Immediately after irradiation PBS was replaced by fresh test medium.

Treatment at the End of the Test Phase (D9):

After treatment the culture medium was removed. The cells were washed with PBS at pH 6.8 and melanin was extracted by adding NaOH-DMSO solution. Melanin extracts were heated at 80° C. for 2 hours. After cooling, aliquots were added in 96-well microplates. Optical density (OD 450 nm) was recorded at 450 nm with a microplate reader (Dynatech MR 5000).

Synthetic melanin standards (Sigma) were incubated at the same conditions. This standard curve allowed the transformation of OD 450 nm into the Melanin Unit Equivalent in each well.

Melanin content was measured by absorbance at 405 nm in control and treated melanin extracts. Results were expressed as μg melanin per well determined from the standard curve: DO(450 nm)=f([melanin]).

Results:

Intracellular melanin content was measured on human normal melanocytes (line M99-1H6) after the formerly described treatment and UVB exposures of cultures. Three concentrations of each tested sample were studied: 1 μg/ml, 5 μg/ml and 10 μg/ml. The cell number assessment (by Red Neutral Uptake Method) showed the absence of toxicity of the product at the three tested doses.

The melanin content (μg of melanin per well) was corrected by the cellular density of each respective culture (number of cells per well) in order to express the “Pigmentation level” of cells (expressed in pg melanin/cell). The pigmenting activity (PA) of the test product was calculated according the formula:

$$P A ( \% ) = \frac{\begin{matrix}{( Melanin content / cell )}_{Treated} - \\ {( Melanin content / cell )}_{Control}\end{matrix}}{{( Melanin content / cell )}_{Control}} \times 100$$

and the results are reported in Table 2.

TABLE 2

| | | | | Melanin content | |
|---|---|---|---|---|---|
| Test | | | | (pg melanin/well) | PA (%) |
| Control | Without UV | Control− | | 50.34 +/− 0.93 | — |
| | With UV | Control+ | | 74.10 +/− 1.59 | — |
| Buddleja | With UV | 1 | μg/ml | 61.14 +/− 0.87 | −17% (p < 0.01) |
| extract | | 5 | μg/ml | 58.85 +/− 0.47 | −21% (p < 0.01) |
| | | 10 | μg/ml | 59.61 +/− 0.00 | −20% (p < 0.01) |
| Luteolin | With UV | 1 | μg/ml | 57.56 +/− 0.0 | −22% (p < 0.01) |
| | | 5 | μg/ml | 55.57 +/− 0.0 | −25% (p < 0.01) |
| | | 10 | μg/ml | 54.84 +/− 1.56 | −26% (p < 0.01) |
| Verbascoside | With UV | 10 | μg/ml | 59.16 +/− 1.56 | −20% (p < 0.01) |
| | | 50 | μg/ml | 58.32 +/− 4.44 | −21% (p < 0.01) |
| | | 100 | μg/ml | 57.80 +/− 1.63 | −22% (p < 0.01) |

A significant decrease of intracellular melanin content was observed when melanocytes, subjected to quite daily UVB exposures, were incubated with the three test products (Table 2). The tested product had a significant inhibitory effect on melanogenesis in UVB-stimulated melanocytes; the decrease of melanin content was significant (p≦0.01, Student's t test) in comparison to the irradiated control culture and equivalent for each product at the 3 concentrations.

### EXAMPLE 4

### Skin Unifying Serum

A formulation was prepared using the ingredients set forth in Table 3:

TABLE 3

| INCI Name | Amount(g) |
|---|---|
| Glycerin | 3.00 |
| Propylene Glycol | 2.00 |
| Buddleja Axillaris Leaf Extract according to example 1 | 1.00 |
| Octocrylene | 0.20 |
| Phenoxyethanol + Methylparaben + Ethylparaben + | 0.80 |
| Propylparaben + Isobutylparaben | |
| Carbomer | 0.50 |
| Tetrasodium EDTA | 0.10 |
| Sodium hydroxide | qs pH 5.5-6 |
| Water | qs 100 g |

### EXAMPLE 5

### Anti-Pigmentation Cream

A formulation was prepared using the ingredients set forth in Table 4.

TABLE 4

| INCI Name | Amount(g) |
|---|---|
| Cetyl Alcohol (and) Glyceryl Stearate (and) PEG-75 | 6.00 |
| Stearate (and) Ceteth-20 (and ) Sterateh-20 | |
| PPG-115 Stearyl Ether | 4.00 |
| Glycerin | 3.00 |
| Propylene Glycol Dipelargonate | 2.50 |
| Isohexadecane | 2.40 |
| Dicaprylyl Ether | 1.20 |
| Isopropyl palmitate | 1.20 |
| Propylene Glycol | 1.00 |
| Buddleja Axillaris Leaf Extract according to example 1 | 0.50 |
| Dimethicone | 0.50 |
| Phenoxyethanol (and) Iodopropynylbutylcarbamate | 0.50 |
| Sodium Bisulfite | 0.15 |
| Disodium EDTA | 0.05 |
| Ascorbic acid | 0.05 |
| Sodium hydroxide | qs pH 5-6 |
| Water | qs 100 g |

### EXAMPLE 6

### Evaluation on Volunteers

The inhibiting effect of a formulation containing 0.5% of the Buddleja axillaris extract (the formulation of Example 5) was investigated using Asian volunteers by evaluating cutaneous pigmentation induced by UVA irradiation, versus a reference product. The reference was a cream comprising the same excipient as the formulation of Example 5 but with 1% arbutin as the listed active ingredient. The protocol is set forth in Table 5

TABLE 5

| | Volun- | | | Volunteers |
|---|---|---|---|---|
| | teers | Volunteers | | included |
| | in- | completing | Phototypes | in data |
| Treatment | cluded | study | Distribution | analysis |
| 0.5% Buddleja | 8 | 7 | 5 Photoype III | 6 |
| axillaris | | | 2 Phototype IV | |
| extract cream | | | | |
| Cream with | 8 | 8 | 6 Photoype II | 8 |
| 1% Arbutin | | | 2 Phototype IV | |

For each product, this was an open, intra-individual study; each subject was his or her own control. The study was conducted in parallel groups (one group by product). Emulsions were applied twice-daily (morning and evening) beginning 14 days before the test started and during the 10 days that the study lasted (from D0 to D10). The creams were applied to the treated zone on the back under normal conditions of use, i.e., the emulsion was applied by a third person by massage until product penetration. A mask was used in order to ensure the proper alignment of the administered emulsion to the test zone. Treatment allocation and the side of application of the product (right/left) were randomized. The skin color measurements were performed at D0, D2, D4 and D10. Irradiations (1 MPD=Minimum Pigmenting Dose) were applied at D0, D2 and D4.

UVA Irradiations were performed with a xenon lamp with a short arc (Arquatiel Idem 2000, spectrum: 320-400 nm) equipped with filters for IR and Visible Radiations eliminations.

Evaluation of the cutaneous pigmentation evolution (bronzing intensity induced by UV with and without product application) was done by colorimetric measurements using a CR321 Minolta® Chromameter®. The Chromameter® converted colors to a digital code composed of three parameters: “L*” for clarity (from dark to light), “a*” for the green-to-red spectrum, and “b*” for the blue-to-yellow spectrum. “a*” and “b*” are chrominance parameters, and “L*” is a luminance parameter. This instrument is commonly used in cosmetics and medicine to measure skin color.

The most characteristic chromomeric parameters of pigmentation are yellow color (b*) and luminance (L*). An increase in luminance L* reflects a diminution of the pigmentation intensity. An increase of b* characterizes an increase of the yellow component of the skin and then a decrease of the pigmentation intensity.

Both parameters can be exploited through the calculation of ITA° (Individual Topologic Angle) which defines the skin pigmentation degree of a subject integrating the clearness (L*) and the melanization parameter (b*) according to the following formula:

ITA°=(Arc TAN((L*−50)/b*))×180/π

An increase of ITA° characterizes a decrease of the pigmentation intensity.

The variations (Δ) of the calorimetric parameters L*, b* and ITA° on the treated and non-treated zones were calculated according to the following formulas:

Δ=(TZti−TZt0)−(NTZti−NTZt0)

where:
TZ: value obtained on the treated zone.
NTZ: value obtained on the non-treated zone.
t0: before product application.
ti: at each measurement time after product application.

Variations in arbitrary unit (A.U.) or in degree (°) obtained for each volunteer, as well as the descriptive statistics, are presented in Tables 6 and 7.

TABLE 6
Variations of cutaneous color after UV irradiations and after repeated
applications of the product containing 0.5% of Buddleja axillaris
extract (T1). Comparison with a non-treated zone (NT)

| | Studied | Raw variations | Volunteers with | |
|---|---|---|---|---|
| Kinetics | param- | T1/NT | an inhibition of | Significance |
| (n = 7) | eters | (moy ± SEM) | pigmentation | (ANOVA) (n = 6) |
| D2/D0 | L* | +2.01 ± 0.64 | 6/7 | Yes (p = 0.011) |
| | b* | −0.81 ± 0.31 | 6/7 | Limit (p = 0.085) |
| | ITA° | +7 ± 2 | 6/7 | Yes (p = 0.007) |
| D4/D0 | L* | +1.38 ± 0.55 | 6/7 | Yes (p = 0.009) |
| | b* | −0.51 ± 0.41 | 5/7 | Limit (p = 0.093) |
| | ITA° | +5 ± 2 | 5/7 | Yes (p = 0.006) |

After bronzing induction by UVA irradiation, the zone treated by the emulsion containing 0.5% of Buddleja axillaris extract was significantly less pigmented than the non-treated zone for the majority of the volunteers (Table 6). An increase of ITA° was observed: +7° and +2° on D2 and D4 respectively in comparison With the non-treated zone (p=0.007 and 0.006). There was no significant difference between the treated and non-treated zones on D10. However, it seems that for some volunteers the bronzing began to disappear six days after the last irradiations, which could explain this result. The product containing 0.5% of Buddleja axillaris extract significantly inhibited the cutaneous pigmentation induced by UVA until D4.

TABLE 7
Variations of cutaneous color after UV irradiation and after
repeated applications of the product containing 1% of arbutin
(T2) and comparison with a non-treated zone (NT)

| | | | Number of | |
|---|---|---|---|---|
| | Studied | Raw variations | volunteers with | |
| Kinetics | param- | T/NT | an inhibition of | Significance |
| (n = 8) | eters | (moy ± SEM) | pigmentation | (ANOVA) (n = 8) |
| D2/D0 | L* | +0.32 ± 0.48 | 4/8 | No (p = 0.389) |
| | b* | −0.38 ± 0.34 | 5/8 | Yes (p = 0.019) |
| | ITA° | +2 ± 2 | 4/8 | No (p = 0.220) |
| D4/D0 | L* | +0.48 ± 0.34 | 5/8 | No (p = 0.534) |
| | b* | −0.82 ± 0.37 | 7/8 | No (p = 0.278) |
| | ITA° | +4 ± 1 | 7/8 | Limit (p = 0.060) |

After two UVA irradiations (D4), the zone treated by the product containing 1% of arbutin was less pigmented than the non-treated zone for the majority of the volunteers (increase of ITA° of +4°) in comparison with the non-treated zone for 7 volunteers out of 8, with p=0.060, Table 7). After one and three irradiations (D2 and D10), the inhibiting effect was less important (increase of ITA° of +2° on 4 volunteers out of 8, p=0.220 and 0.363 respectively, Table 7). The product containing 1% arbutin tended to inhibit the cutaneous pigmentation induced by UVA for the majority of the volunteers (variations at the limit of significance on D4).

### EXAMPLE 7

### Ex Vivo Activity on Dendrites Formation of Melanocytes

The anti-pigmenting activity of an emulsion containing 0.5% of a Buddleja axilaris extract (from Example 5, P) versus excipient (E) was evaluated. Biopsies from abdominal plastic surgery (27-year-old woman, Phototype II/III) were used in this ex vivo experiment. The biopsies were cultured in a specific survival explants medium BEM (BIO-EC's Explants Medium) and were shared out according to their specific treatment:

The following notations are used in this example:

| Property | Notation |
|---|---|
| Control | C |
| Unrayed Control explants | C − UV |
| 0.5% Buddleja axilaris extract (from Example 5) | P |
| Excipient | E |
| Rayed Control explants | C + UV |
| Unirradiated explants, treatment with the | P − UV |
| excipient + 0.5% of Buddleja axillaris extract | |
| Unirradiated explants, treatment with the | E − UV |
| excipient | |
| Irradiated explants, treatment with the | P + UV |
| excipient + 0.5% of Buddleja axillaris | |
| extract | |
| Irradiated explants, treatment with the excipient | E + UV |

2 mg of the product (P, E) were applied topically to the explant and spread with a small spatula. These applications were performed on each treated explant at Day 0 (D0), D1, D2, D3, D4, D5, D6, D7 and D8.

The explants C-UV, P-UV and E-UV were not irradiated. The explants C+UV, P+UV and E+UV received daily irradiations (UVA: 2.25 J/cm², UVB 0.135 J/cm²). Irradiations were performed 2 hours before the topical applications of the excipient or the excipient +0.5% of the extract. During irradiations the culture medium of the explants was changed to HBSS buffer after the explants are put back in BEM (BIO-EC's Explants Medium). The explants were taken off for histological study at D3, D6 and D9. Each time explants were cut in 3 parts. One part was fixed in formol and the other parts were frizzed at −80° C.

A general morphological study was performed on the formol-fixed explants after dehydration, paraffin impregnation and staining according to the Masson's method.

DOPA-oxidase reaction explants were treated according to the Laidlaw and Blackberg method. This technique enables an in situ assessment of the product activity on tyrosinase.

The results showed that at D0 the Melanocytes were moderately DOPA-positive. At D3 melanocytes in P+UV explants were slightly DOPA-positive, clearly less than positive than the untreated control. Their dendricity was slightly reduced. In E+UV explants melanocytes were clearly DOPA-positive and slightly dendritic. At D6 all the explants (C+UV, P+UV, E+UV) were clearly DOPA-Positive. While melanocytes in E+UV were clearly dendritic, dendricity for P+UV cells was slightly reduced. At D9 melanocytes in P+UV were slightly DOPA-positive, clearly less than in C+UV. Dendricity was clearly reduced. Melanocytes in P2+UV were clearly DOPA-positive and very clearly dendritic. The P1 product induced a decrease of the DOPA-positivity. This effect was more observable after 9 days. This example shows reduction of the dendricity of melanocytes as was observed from the 6th day and after UV irradiation. The product E did not induce any change either in the DOPA positivity or in the melanocytes dendricity.

Under these operative conditions, compared to what is observed with the untreated explants, the results indicated that the product P1 (excipient +0.5% of Buddleja axillaris extract) had a clear anti-pigmenting/lightening activity.

What is claimed is:

1. A combination comprising verbascoside and luteolin.

2. The combination of claim 1, wherein at least one other active substance employed for dermatological use.

3. The combination of claim 2, wherein said at least one other active substance comprises a substance for lightening the skin, preventing or treating spots.

4. The combination of claim 2, wherein said at least one other active substance is selected from the group consisting of hydroquinone, tretinoin, topical steroids, azelic acid, kojic acid, arbutin, licorice extracts and mixtures thereof.

5. A plant extract comprising a combination of verbascoside and luteolin.

6. The plant extract of claim 5, wherein said verbascoside comprises at least about 10% by weight of said extract and luteolin comprises up to about 5% by weight of said extract.

7. The plant extract of claim 5, wherein the extract is an extract of Buddleja axillaris.

8. A method for treating skin comprising administering a therapeutic amount of verbascoside and luteolin to a patient in need of treatment.

9. The method of claim 8, wherein said method comprises topical administration of said verbascoside and luteloin.

10. The combination of claim 1, wherein said combination comprises a liquid solution, an ointment or a cream.

11. The combination of claim 10, wherein said verbascoside comprises from about 0.0001% by weight to about 10% by weight of said combination and said luteolin comprises from about 0.00001% by weight to about 1% by weight of said combination.
